(19) United States
(12) Patent Application Publication
(10) Pub. No.: US 2007/0259988 A1
(43) Pub. Date: Nov. 8, 2007

(54) Biomedical bond enhancer

(75) Inventors: Gregory J. Dorsman, Costa Mesa, CA (US); Nicholas D. Breslin, Costa Mesa, CA (US)

Correspondence Address: FOLEY & LARDNER LLP, 1530 PAGE MILL ROAD, PALO ALTO, CA, 94304, US

(73) Assignee: Danville Materials, Inc.

(21) Appl. No.: 11/541,421

(22) Filed: Sep. 29, 2006

Related U.S. Application Data
(60) Provisional application No. 60/722,220, filed on Sep. 30, 2005.

(51) Int. Cl. A61K 6/00; B31B 1/60

(52) U.S. Cl. 523/118; 156/60

(57) ABSTRACT

The invention provides methods and compositions to enhance the bond between light-cure bonding agents and self-cured or dual-cured resin-based composites used in biomedical applications, especially dental procedures to restore teeth. The invention is a bond enhancer in the form of a solution that can be applied after application to dental substrates of a light-cure adhesive and prior to use of a self-cure or dual-cure composite to improve the compatibility between the adhesive and composite as evidenced by increased bond strength. The solution contains at least one polymerization accelerator, at least one polymerizable monomer or prepolymer, and optional polymerization inhibitors in an organic solvent or mixture of solvents.

### CROSS-REFERENCE TO RELATED APPLICATION

This application claims the benefit of U.S. Provisional Application No. 60/722,220, filed Sep. 30, 2005, which is herein incorporated by reference.

### BACKGROUND OF THE INVENTION

Resin cements have found widespread use for applying indirect restorations to teeth due to their low water solubility and excellent wear properties (El-Mowafy, et al., Journal of Prosthetic Dentistry 76:524-9 (1996); El-Mowafy, et al., Operative Dentistry 19:176-82 (1994)), however, by themselves the current cements do not adhere well enough to tooth or restoration surfaces to prevent micro-leakage or even loss of the restoration. Micro-leakage allows both oral fluid and bacterial infiltration, and could result in marginal staining, post-operative sensitivity, pulpal inflammation and the onset of secondary caries. Dental adhesives and surface treatments, including etchants, conditioners, cleansers, and primers, have been developed that improve the adherence of cements to tooth and restoration surfaces. The adhesives take the form of light-cure, self-cure, and dual-cure resins that contain adhesion promoters and optionally, fillers. The light-cure adhesives generally come in a single package and do not require mixing prior to use. They are applied to the treated surfaces in a thin layer and require application of light, usually visible light in the blue range, in order to harden and promote adhesion to the surface. The self-cure and dual-cure adhesives are generally in the form of two parts packaged separately during storage that require mixing just prior to application to the surface. The dual-cure adhesive can optionally be cured using visible light. The advantage of light-cure adhesives is that they are presented in only one part and do not require mixing prior to use. Moreover, it has been reported that light-cure formulations provide improved adhesion over self-cure or dual-cure formulations, all other components being the same. The advantage of self-cure adhesives is that they do not require light-curing in order to function. This makes them valuable in situations where it is not possible for light to penetrate. However, the dentist may need to wait for the curing reaction to take place before moving on to other steps in the tooth restoration process. The advantage of dual-cure adhesives is the same as for self-cure, with a further advantage that the material can be made to set or polymerize with light on command, allowing further processing. There have been reports, however, that dual-cure adhesives do not perform as well as self-cure adhesives when no light is applied for curing (McCabe and Rusby, British Dental Journal 179:333-6 (1994)).

Resin cements for indirect dental procedures need to harden or polymerize in the absence of light since many restorations include a metal substructure that light does not penetrate or are so thick or deep that the energy of light reaching all areas of the cement has been diminished by absorption within the tooth, the prostheses, and/or the cement itself. The amount of light reaching an inner layer can be predicted using Beer's Law if one knows the power of the incident light, the thickness of each layer, and the absorption coefficients of the restorative materials. Since some of these values are not known, when affixing indirect restorations to teeth, it is safest to use self-cure or dual-cure cements rather than light-cure resin cements. Light-cure resin cements do find value in other applications due to their low water solubility, good color stability, and excellent wear resistance. They are best in bonding veneers and small inlays or onlays that are generally made of ceramic or composite with relatively high translucency that allows a great portion of the light to pass through to affect curing. Like with the adhesives, the advantage of dual-cure cements is that they can be hardened at the restoration periphery or margin by application of light of the correct wavelength and power so that the dentist can finish the restoration. Finishing means that excess restorative material is removed, the prosthesis and its cement are shaped to provide contour, proper function, and a gap between the restored tooth and other teeth that contact it, and the restoration is polished, especially at the margins, so that it reflects light like surrounding dentition and resists accumulation of plaque or oral contaminants.

Prior to almost any tooth restoration procedure, the tooth is cut to remove infected structure and shaped to accept the restorative material and/or prosthesis. When using a resin-based restorative, an adhesive is next applied to the cut tooth surface. New generations of resin-based adhesives may be applied directly to the surface, but most forms generally require preparation of the tooth surface prior to their application. One method of preparation is to apply an etchant, an aqueous solution or gel containing an acid, typically phosphoric acid in the range of 10% to 40% of the whole, to the tooth for a short period of time, typically 5 to 60 seconds. The etchant is rinsed away and the tooth surface dried or partially dried depending on the composition of the adhesive that is applied next. The etchant method of preparation has an advantage that it is most effective on enamel, the outer layer of teeth, because it opens deeper tags that provide micromechanical retention. However, when etchants are used on dentin they require careful control of the moisture removal in order to provide good adhesion. Apparently, the inorganic matrix of dentin is removed by dissolution by acid, leaving fibrous collagen protein at the surface (Nakabayashi, et al., Journal of Esthetic Dentistry 3:133-138 (1991)). As rinse water is removed to dryness, the protein fibers collapse into a dense mat that is not easily adhered to (Van Meerbeek, et al., Journal of Dental Research 77:50-59 (1998)). At intermediate levels of moisture, the adhesive is still able to penetrate the fibers to form an inter-diffusion zone (Van Meerbeek, et al., Journal of Dental Research 72:495-501 (1993)). In the second method, a primer is applied to the etched tooth prior to the adhesive. After the etchant or conditioner is rinsed off, a primer containing one or more hydrophilic resin monomers is applied. Primer molecules contain two functional groups: the hydrophilic group has an affinity for dentinal surfaces and the hydrophobic group has an affinity for resin. The primer wets and penetrates the collagen meshwork, raising it almost to its original level, and also increases the surface energy, hence the wettability of the dentinal surface. Unfilled resin is applied to and penetrates the primed dentin, copolymerizing with the primer to form a hybrid layer with collagen (Morcari, et al., Brazilian Dental Journal 13:33-38 (2002)). A third method of preparation involves a more recent development, which is to apply a self-etch primer, an aqueous solution containing an organic acid, preferably an acid that can participate in polymerization with the adhesive, to the tooth and then to remove only the water and/or solvent from the primer layer, optionally light curing the remaining layer, and then applying the adhesive next. Application of the adhesive without surface treatment is ideal, but the full clinical effectiveness has yet to be studied. When teeth are cut prior to etchant application, a smear layer forms that blocks the tubules in dentin. When etchants are used on dentin, they open some of the tubules so that there is direct communication to the pulp chamber and the dental nerve, which, in deeper dentin, may cause pain to the patient or allow bacterial ingress if not completely sealed. In this case the use of self-etch primers may be preferred. There are reports that self-etching primers may reduce the occurrence of post-operative pain by causing only partial hydroxyapatite dissolution that leaves the tubule smear plugs intact. The new generations of resin-based adhesives combine the functions of the etchant, primer, and adhesive into one composition. In these and most other adhesives, the active ingredient is a multifunctional molecule having one or more acid groups at one end to interact with the tooth surface and one or more polymerizable groups at the other end that can interact with polymerizable materials placed onto them.

Resin-based adhesives with or without etchant and/or primers are currently used with resin cements to secure indirect restorations and with more highly filled composites for core build-ups. While light-cure adhesives are preferred for their strength and ease of application, there have been reports of incompatibility between them and self-cure or dual-cure resin cements leading to poor bond strengths and low retention (Swift, et al., American Journal of Dentistry 14(3):137-40 (2001)). Several manufacturers developed dual-cure activators intended to be mixed with their light-cure adhesives to overcome incompatibility between light-cure adhesives and self-cure core build-up composites (CRA Newsletter April 2003, http://www.cranews.com/additional_study/2003/03-04/page2.htm). Several possible reasons for this incompatibility have been suggested. When resin-based adhesives are polymerized, an oxygen-inhibited layer remains containing the active ingredient molecule having one or more acid groups. This unpolymerized layer mixes with the self-cure composite during its placement. The acid of the adhesive reacts with the aromatic tertiary amine accelerator of the dual-cure or self-cure composite cement effectively retarding its cure (Sanares, et al., Dental Materials 17:542-56 (2001)). The degree of retardation is related to the thickness of the oxygen-inhibited layer and the concentration of the acid and can be only partially offset by the addition of sodium benzenesulfinate (Suh, et al., Journal of Adhesive Dentistry 5:267-82 (2003)). Another factor for the light-cure adhesive/auto-cure composite incompatibility is that water transports from the hydrated dentin to the adhesive-composite interface during the time it takes for the setting reaction to be completed (Tay, et al., American Journal of Dentistry 17:271-8 (2004)). The magnitude of the effect water transport has on adhesion of auto-cure composites to light-cure adhesives may relate to the rate of composite polymerization compared to the rate of transport. The polarity of adhesive monomers affects the level of hydrophilicity of the system and hence its ability to transport water.

### SUMMARY OF THE INVENTION

The invention is a bond enhancer in the form of a solution that can be applied after application to dental substrates of a light-cure adhesive and prior to use of a self-cure or dual-cure composite to improve the compatibility between the adhesive and composite as evidenced by increased bond strength. The solution contains at least one polymerization accelerator, at least one polymerizable monomer or prepolymer, and optional polymerization inhibitors in an organic solvent or mixture of solvents.

One aspect of the invention provides bond enhancers for placement between a light-cure adhesive and a self-cure or dual-cure composite, said enhancer comprising at least one polymerizable monomer or prepolymer and a polymerization accelerator in an organic solvent or mixture of organic solvents. The bond enhancer may further comprise a polymerization inhibitor. Preferably, the organic solvent is selected from the group consisting of acetone, acetonitrile, benzyl alcohol, 1-butanol, 2-butanol, isobutanol, tert-butanol, chloroform, ethanol, ethyl acetate, ethyl methyl ketone, methyl acetate, 1-propanol, and 2-propanol, and mixtures thereof. A preferred polymerizable monomer is a methacrylate compound, such as a Bowen monomer, bis-GMA, ethoxylated Bis-GMA, triethyleneglycol dimethacrylate, or mixtures thereof.

Another aspect of the invention provides methods of enhancing a bond to a biomedical surface comprising: (a) applying a light-cure adhesive to a biomedical surface; (b) applying a bond enhancer to the light-cure adhesive, wherein the bond enhancer comprises at least one polymerizable monomer or prepolymer and a polymerization accelerator in an organic solvent or mixture of organic solvents; and (c) applying a self-cure or dual-cure composite to the bond enhancer prior to the self-cure or dual-cure composite being fully cured. The method may further comprise applying an etchant and/or primer to the biomedical surface prior to applying the light-cure adhesive. In certain embodiments, at least two of the etchant, primer and light-cure adhesive are combined into a single composition.

One aspect of the invention provides methods of bonding a light-cure adhesive to a self-cure or dual-cure composite comprising: (a) applying a bond enhancer to a layer of light-cure adhesive, wherein the bond enhancer comprises at least one polymerizable monomer or prepolymer and a polymerization accelerator in an organic solvent or mixture of organic solvents; and (b) applying the self-cure or dual-cure composite to the bond enhancer prior to the self-cure or dual-cure composite being fully cured. The bond enhancer may be applied to the layer of light-cure adhesive before the light-cure adhesive is cured or after the light-cure adhesive has been exposed to a light source.

Yet another aspect of the invention provides methods of enhancing a bond between two biomedical surfaces comprising: (a) applying a light-cure adhesive to a first biomedical surface; (b) applying a bond enhancer to the light-cure adhesive, wherein the bond enhancer comprises at least one polymerizable monomer or prepolymer and a polymerization accelerator in an organic solvent or mixture of organic solvents; (c) applying a self-cure or dual-cure composite to a second biomedical surface; and (d) fitting the second biomedical surface to the first biomedical surface, prior to the self-cure or dual-sure composite being fully cured.

An aspect of the invention provides methods of adhering two biomedical surfaces comprising: (a) applying a light-cure adhesive to a first biomedical surface; (b) applying a bond enhancer to the light-cure adhesive, wherein the bond enhancer comprises at least one polymerizable monomer or prepolymer and a polymerization accelerator in an organic solvent or mixture of organic solvents; (c) applying a self-cure or dual-cure composite to a second biomedical surface; and (d) fitting the second biomedical surface to the first biomedical surface, prior to the self-cure or dual-sure composite being fully cured.

An aspect of the invention provides kits for adhering dental surfaces comprising: (a) a bond enhancer comprising at least one polymerizable monomer or prepolymer and a polymerization accelerator in an organic solvent or mixture of organic solvents; and (b) a light-cure dental adhesive system, or a self-cure or dual-cure dental composite. In preferred embodiments, the bond enhancer further comprises a polymerization inhibitor. In certain preferred embodiments, the kit comprises both a light cure dental adhesive system and a self-cure or dual-cure dental composite. The light-cure dental adhesive system may comprise: 1) a light-cure adhesive; 2) a primer and light-cure adhesive suitable for placement on etched tooth surfaces; 3) a self-etch primer and a light-cure adhesive suitable for placement directly on tooth surfaces; or 4) a light-cure adhesive suitable for placement directly on tooth surfaces without prior priming or etching.

### DETAILED DESCRIPTION OF THE INVENTION

Overview

The invention provides methods and compositions to enhance the bond between light-cure bonding agents and self-cured or dual-cured resin-based composites used in biomedical applications, especially dental procedures to restore teeth. The invention is a bond enhancer in the form of a solution that can be applied after application to dental substrates of a light-cure adhesive and prior to use of a self-cure or dual-cure composite to improve the compatibility between the adhesive and composite as evidenced by increased bond strength. The solution contains at least one polymerization accelerator, at least one polymerizable monomer or prepolymer, and optional polymerization inhibitors in an organic solvent or mixture of solvents.

Definitions

The term “cured,” as used in the specification and the appended claims, refers to a composition that has been sufficiently polymerized so as to be sufficiently hardened and non-pliable so that it is not malleable or readily deformable. Thus though a completely polymerized composition represents an example of a “cured” material, a composition that is substantially polymerized so as to resist deformation, even if it is capable of some further polymerization, may still be considered to be “cured.”

Methods of Enhancing Bonds to Biomedical Surfaces

This invention relates to methods used to enhance the bond between light-cure bonding agents and self-cured or dual-cured resin-based composites used in biomedical applications, especially dental procedures to restore teeth. The composite can be a resin cement used to permanently affix indirect restorations (prostheses) to teeth or a material that builds up the core of the tooth or repair hard tissue of the human body. Indirect restorations can include prostheses manufactured to replace part of the tooth's missing anatomy or pins or posts inserted into the root canals of teeth to provide improved retention of other materials used to build up or restore the tooth or bone to a functional form.

The problem of incompatibility between light-cure adhesives and self-cure composites has been well documented. Older generations of adhesives did not appear to exhibit this problem since the adhesive itself was typically made up of monomers that were similar to the monomers found in the self-cure composites layered over them. Therefore, the oxygen inhibition layer of the adhesive did not contain significant acid-functional monomers that would retard the curing reaction of the self-cure composite. Also, since the monomers were less hydrophilic than current simplified-step light-cure adhesive monomers, the cured layer had the effect of inhibiting the transport of water to the adhesive-composite interface where it could affect the bonding area. Simplified step adhesives, to which preferred embodiments of this invention applies, involve the steps of etching, priming, and applying adhesive where at least two of the steps are performed by a single composition. For instance, the etchant and primer might be combined with the adhesive separate, the primer and adhesive combined with the etchant separate, or all three combined into one composition. Generally, it is these simplified-step light-cure adhesive systems that are incompatible with self-cure and dual-cure dental composites. The invention is used after completion of the steps of these adhesive systems to cause them to become compatible with self-cure composites. In response to reports of adhesive/self-cure composite incompatibility, some manufacturers introduced dual-cure adhesives, however, this adds an extra step of mixing and the incompatibility was not entirely relieved. Notably, the bond enhancer of the present invention is not mixed with the adhesive, but rather applied over the adhesive.

Although much effort has been expended to overcome the problem of incompatibility between light-cure adhesives on biomedical surfaces and self-cure composites, it has surprisingly been found that the present invention also allows improved bonding between self-cure composites and light-cure adhesives applied to other surfaces such as metals, ceramics, and cured composites.

The present invention provides methods of enhancing a bond to biomedical surfaces comprising: (a) applying a light-cure adhesive to a biomedical surface; (b) applying a bond enhancer to the light-cure adhesive, wherein the bond enhancer comprises at least one polymerizable monomer or prepolymer and a polymerization accelerator in an organic solvent or mixture of organic solvents; and (c) applying a self-cure or dual-cure composite to the bond enhancer prior to the self-cure or dual-cure composite being fully cured.

By applying a bond enhancer to the cured adhesive layer its oxygen-inhibited layer is essentially dissolved and mostly carried away by the solvent, which is present in excess by virtue of the fact that even one drop will flood repair surfaces typical in dental or osseous surgeries. While not being limited to a theory of the invention, it is believed that when the oxygen-inhibited (unpolymerized) layer is removed or greatly dispersed by the solvent, the remaining surface is rich in unreacted functionality that is available for incorporation into a polymer film made of polymerizable organic material that is placed onto it during the act of performing the method of the invention. The unpolymerized layer of the light-cure adhesive can be dispersed by the solvent by the mere act of applying a drop of the bond enhancer to the surface of the cured adhesive or can be manually dispersed stirring or swabbing the bond enhancer across the adhesive surface. In a preferred embodiment of the invention, the light-cure adhesive comprises an acidic or hydrophilic monomer, more preferably an acidic monomer

Another surprising observation using the present invention is that it is not necessary to cure the light-cure adhesive prior to application of the bond enhancer when next applying a self-cure or dual-cure composite, in order to obtain an improvement in the bond strength of the system. The adhesive may be intimately confined to the surface either by chemical or physical action so that the solvent in the bond enhancer does not remove more than that part that is needed to effect adhesion when both layers cure by virtue of the contact with the overlying self-cure composite.

Compositions of the invention contain polymerizable organic material that comes into intimate contact with the adhesive surface as the solvent is removed, forming a film, particularly after removal of most or all of the organic solvent, preferably by evaporation. The film is comprised of the polymerizable monomer or prepolymer of the bond enhancer, the accelerator of the bond enhancer, optional polymerization inhibitor, and, potentially, remnants of the oxygen-inhibited layer of the light-cure adhesive that have been dispersed into the bond enhancer upon application.

The effectiveness of the bond enhancer of the invention is dependent on its ability to polymerize quickly upon being contacted by the mixed self-cure or dual-cure composite. By doing so the polymer film, being made of relatively hydrophobic material, slows or stops the transport of moisture from the tooth to the adhesive-composite interface. Some of the initiator is available at the surface of the mixed mass of the as yet uncured self-cure composite or becomes available by mixing with the bond enhancer film during its placement during restorative procedures.

The free radical initiator of the self-cure composite and the polymerization accelerator in the bond enhancer initiate a polymerization reaction that involves the surface of the adhesive, the bond enhancer, and the self-cure composite so that excellent strength is obtained through the interface.

Bond Enhancer Compositions

This invention relates to compositions used to enhance the bond between light-cure bonding agents and self-cured or dual-cured resin-based composites used in biomedical applications, especially dental procedures to restore teeth. The bond enhancer of the invention comprises at least one polymerizable monomer or prepolymer and a polymerization accelerator in an organic solvent or mixture of organic solvent. Optionally, the bond enhancer may further comprise a polymerization inhibitor.

Any known polymerizable monomer or prepolymer that can be used in a dental composite material and is soluble in solvents of the invention can be utilized in the present invention without any limitation. The polymerizable monomer may be a methacrylate compound, preferably a Bowen monomer, bis-GMA, an alkyl dimethacrylate, ethoxylated Bis-GMA, diurethane dimethacrylate, or mixtures thereof. Those familiar with the art may substitute acrylates or alkyl acrylates for methacrylates, as desired. The polymerizable monomer or prepolymer is typically a mixture of monomers and may be present in the bond enhancer in an amount that is between about 0.5% w/w and about 90% w/w of the solution. More preferably, the polymerizable organic material is present in an amount of about 2% w/w to about 10% w/w.

Monomers typically are methacrylate compounds such as a Bowen monomer (such as those described in U.S. Pat. Nos. 3,066,122; 3,179,623; and 3,194,784 to Bowen, incorporated herein by reference), Bis-GMA, and related backbone and diluent species, but are not necessarily limited to this chemical class. The polymerizable organic material can be made of any species that can be made to harden either by mixing two component parts or by activating with external radiation such as visible or ultraviolet light.

Preferred monomer mixtures are more hydrophobic than Bis-GMA and hydroxyethylmethacrylate (HEMA) in a one to one weight ratio. Such mixtures will generally not contain more than 10% w/w monomers with an acid functionality and preferably less than 5% w/w. In certain preferred embodiments of the invention, the monomer mixture comprises at least one backbone monomer, such as bis-GMA, and at least one diluent monomer.

One preferred embodiment of the invention comprises methacrylates that comprise monofunctional vinyl monomers. This group includes methacrylates such as methyl methacrylate, ethyl methacrylate, isopropyl methacrylate, hydroxyethyl methacrylate, tetrahydrofurfuryl methacrylate, glycidyl methacrylate, and acrylates corresponding to these methacrylates.

Another preferred embodiment of the present invention comprises methacrylates that comprise bifunctional vinyl monomers. Accordingly, the bond enhancer may comprise methacrylates comprising aromatic compounds, such as 2,2-Bis(methacryloyloxyphenyl)propane, 2,2-bis[4-(3-methacryloyloxy)-2-hydroxypropoxyphenyl]propane (Bis-GMA), 2,2-bis(4-methacryloyloxyphenyl)propane, 2,2-bis(4-methacryloyloxypolyethoxyphenyl)propane, 2,2-bis(4-methacryloyloxydiethoxyphenyl)propane, 2,2-bis(4-methacryloyloxytetraethoxyphenyl)propane, 2,2-bis(4-methacryloyloxypentaethoxyphenyl)propane, 2,2-bis(4-methacryloyloxydipropoxyphenyl)propane, 2-(4-methacryloyloxyethoxyphenyl)-2-(4-methacryloyloxydiethoxyphenyl)propane, 2-(4-methacryloyloxydiethoxyphenyl)-2-(4-methacryloyloxyditriethoxyphenyl)propane, 2-(4-methacryloyloxydipropoxyphenyl)-2-(4-methacryloyloxytriethoxyphenyl)propane, 2,2-bis(4-methacryloyloxypropoxyphenyl)propane, 2,2-bis(4-methacryloyloxyisopropoxyphenyl)propane, and acrylates corresponding to these methacrylates. The methacrylates may also be a diadduct obtained by the addition reaction of a vinyl monomer having an -OH group like such methacrylate as 2-hydroxyethyl methacrylate, 2-hydroxypropyl methacrylate or 3-chloro-2-hydroxypropyl methacrylate, or an acrylate corresponding to these methacrylates and a diisocyanate compound having an aromatic group, such as diisocyanatemethyl benzene or 4,4′-diphenylmethane diisocyanate. The bifunctional vinyl monomer may also comprise an aliphatic compound. Exemplary aliphatic bifunctional vinyl monomers include, but are not limited to, ethylene glycol dimethacrylate, diethylene glycol dimethacrylate, triethyleneglycol dimethacrylate (hereinafter abbreviated as 3G), butylene glycol dimethacrylate, neopentyl glycol dimethacrylate, propylene glycol dimethacrylate, 1,3-butanediol dimethacrylate, 1,4-butanediol dimethacrylate, 1,6-hexanediol dimethacrylate, and acrylates corresponding to these methacrylates; diadducts obtained by the addition reaction of a vinyl monomer having an -OH group like such methacrylate as 2-hydroxyethyl methacrylate, 2-hydroxypropyl methacrylate, 3-chloro-2-hydroxypropyl methacrylate or acrylate corresponding to the methacrylate and a diisocyanate compound such as hexamethylene diisocyanate, trimethylhexamethylene diisocynate, diisocynatemethylcyclohexane, isophorone diisocyanate, or methylenebis(4-cyclohexyl isocyanate).

Another preferred embodiment of the invention utilizes trifunctional vinyl monomers of methacrylates, such as trimethylolpropane trimethacrylate, trimethylolethane trimethacrylate, pentaerythritol trimethacrylate and trimethylolmethane trimethacrylate, and acrylates corresponding to these methacrylates. Bond enhancers of the present invention may also comprise tetrafunctional vinyl monomers, such as pentaerythritol tetramethacrylate and pentaerythritol tetraacrylate; and adducts obtained by the addition reaction of a diisocyanate compound such as diisocyanatemethylbenzene, diisocyanatemethylcyclohexane, isophoronediisocyanate, hexamethylenediisocyanate, trimethylhexamethylenediisocyanate, methylenebis(4-cyclohexylisocyanate), 4,4-diphenylmethanediisocyanate or tolylene-2,4-diisocyanate with a glycidol dimethacrylate.

Other preferred polymerizable components can be substituted acryl amides and methacrylamides. Examples are acrylamide, methylene bis-acrylamide, methylene bis-methacrylamide, diacetone/acrylamide diacetone methacylamide, N-alkyl acrylamides and N-alkyl methacrylamides where alkyl is a lower hydrocarbyl unit. Other suitable examples of polymerizable components are isopropenyl oxazoline, vinyl azalactone, vinyl pyrrolidone, styrene, divinylbenzene, urethane acrylates or methacrylates, epoxy acrylates or methacrylates and polyol acrylates or methacrylates.

Another useful class of compounds effective as polymerizable components is tertiary amines that contain polymerizable functionality. These are suitably obtained by reacting aryl hydroxylamines such as N,N-dihydroxyethyl-p-toluidine or alkyl hydroxylamines like triethanolamine with a diisocyanate compound such as hexamethylene diisocyanate, trimethylhexamethylene diisocyanate, methylcyclohexane diisocyanate, isophorone diisocyanate, or methylenebis(4-cyclohexyl isocyanate) or with an alkyl halide or anhydride having a polymerizable group such as methacroyl chloride or methacrylic anhydride.

Prepolymers for use in the invention may include any suitable monomer that is partially reacted thus creating a larger monomer (prepolymer) of higher molecular weight that is fluid and maintains enough reactivity to polymerize to a solid upon exposure to a polymerization initiator and optional accelerator.

Solvents are chosen that are not harmful to tissues of the human body. Preferably the solvent(s) also evaporate quickly with no more than a mild air stream directed upon the surface. Ideally the solvent(s) forms an azeotrope with water so that the surface is left dry upon evaporation. Suitable solvents include acetone, acetonitrile, benzyl alcohol, 1-butanol, 2-butanol, isobutanol, tert-butanol, chloroform, ethanol, ethyl acetate, ethyl methyl ketone, methyl acetate, 1-propanol, and 2-propanol. Most preferred solvents are absolute ethanol and acetone. The solvent or solvent mixture is present in compositions of the invention at levels from 10% to 99.5% by weight of the whole. More preferably they are present in levels from 70% to 99% by weight, more preferably 90% to 98% of the whole composition.

The effectiveness of the bond enhancer of the invention is dependent on its ability to polymerize quickly upon being contacted by the mixed self-cure or dual-cure composite. By doing so the polymer film, being made of relatively hydrophobic material, slows or stops the transport of moisture from the tooth to the adhesive-composite interface. The self-cure composites contain free radical polymerization initiators, for example, a peroxide in an amount ranging from about 1% to 5%. Typical free radical initiators are lauroyl peroxide, tributylhydroperoxide, and more particularly benzoyl peroxide. Some of the initiator is available at the surface of the mixed mass of the as yet uncured self-cure composite or becomes available by mixing with the bond enhancer film during its placement during restorative procedures.

As the polymerization accelerator, aromatic tertiary amines, aliphatic tertiary amines, sulfinic acids and their salts can be cited as preferred ones. They can be present in the composition of the invention in amounts from 0. 1% w/w to 10% w/w of the whole, more preferably from 0.2% w/w to 5% w/w.

Useful accelerators are reducing agents and Lewis bases such as tertiary amines. Examples of the aromatic tertiary amines include N,N-dimethylaniline, N,N-dimethyl-p-toluidine, N,N-dimethyl-m-toluidine, N,N-diethyl-p-toluidine, N,N-dimethyl-3,5-dimethylaniline, N,N-dimethyl-3,4-dimethylaniline, N,N-dimethyl-4-ethylaniline, N,N-dimethyl-4-i-propylaniline, N,N-dimethyl-4-t-butylaniline, N,N-dimethyl-3,5-di-t-butylaniline, N,N-bis(2-hydroxyethyl)-3,5-dimethylaniline, N,N-di(2-hydroxyethyl)-p-toluidine, N,N-bis(2-hydroxyethyl)-3,4-dimethylaniline, N,N-bis(2-hydroxyethyl)-4-ethylaniline, N,N-bis(2-hydroxyethyl)-4-i-propylaniline, N,N-bis(2-hydroxyethyl)-4-t-butylaniline, N,N-bis(2-hydroxyethyl)-3,5-di-i-propylaniline, N,N-bis(2-hydroxyethyl)-3,5-di-t-butylaniline, n-butoxyethyl 4-dimethylaminobenzoate, (2-methacryloyloxy)ethyl 4-dimethylaminobenzoate, and the like.

Specific examples of the aliphatic tertiary amines include trimethylamine, triethylamine, N-methyldiethanolanine, N-ethyldiethanolamine, N-n-butyldiethanolamine, N-lauryldiethanolamine, triethanolamine, (2-dimethylamino)ethyl methacrylate, N-methyldiethanolamine dimethacrylate, N-ethyldiethanolamine dimethacrylate, triethanolamine monomethacrylate, triethanolamine dimethacrylate, triethanolamine trimethacrylate, and the like.

If a sulfinic acid salt and/or a carboxylic acid salt is used as a promotor together with a tertiary amine reducing agent, good results are often obtained. The kind of sulfinic acid salt is not particularly critical, and known sulfinic acid salts may be used. However, it is ordinarily preferred that a salt of a sulfinic acid bonded to an alkyl or aryl group be used. As especially preferred examples, there can be mentioned alkali metal salts such as lithium, sodium, potassium, rubidium and cesium salts, alkaline earth metal salts such as magnesium, calcium and strontium salts, iron, copper and zinc salts, ammonium salts, tetraalkyl ammonium salts and N,N-dimethyl-p-toluidine salts of benzenesulfinic acid, p-toluenesulfinic acid, o-toluenesulfinic acid, ethylbenzenesulfinic acid, decylbenzenesulfinic acid, dodecylbenzenesulfinic acid, beta-naphthalenesulfinic acid, styrenesulfinic acid, 2,4,6-trimethylbenzenesulfinic acid, 2,4,6-triisopropylbenzenesulfinic acid, chlorobenzenesulfinic acid, and the like.

Any of known metal salts of carboxylic acids can be used as another type of the promotor without any limitation. Furthermore, a monobasic carboxylic acid or a polybasic carboxylic acid such as a dicarboxylic acid or a tricarboxylic acid may be used. In case of a polybasic carboxylic acid, a poly-metal salt such as a di-metal salt is preferably used rather than a mono-metal salt. Preferred examples of alkali metal salts such as sodium and potassium salts, alkaline earth metal salts such as magnesium and calcium salts, iron salts, copper salts, zinc salts and silver salts of aliphatic carboxylic acids such as formic acid, acetic acid, propionic acid, butyric acid, valeric acid, lauric acid, stearic acid, pyruvic acid, methoxyacetic acid and acetoacetic acid, aromatic carboxylic acids such as benzoic acid, phenylacetic acid and salicylic acid, hydroxycarboxylic acids such as glycolic acid and lactic acid, dicarboxylic acids such as oxalic acid, succinic acid, adipic acid, tartaric acid, maleic acid, fumaric acid and phthalic acid, and vinyl carboxylic acids such as acrylic acid, methacrylic acid and vinylacetic acid can be mentioned as specific examples of the carboxylic acid salt component of the composition of the present invention.

Aromatic tertiary amines and sulfinic acid salts are preferred accelerators. N,N-di(2-hydroxyethyl)-p-toluidine and sodium benzenesulfinate are more preferred while a combination of these two is most preferred.

In the present invention, the amounts of polymerization accelerators in the form of promotors and/or reducing agents are not critical, and they are appropriately determined according to methods known to those of ordinary skill in the art in view the polymerizable vinyl monomer present in the compositions. Ordinarily, polymerization accelerators are used in an amount of about 1 to 200% by weight based on the polymerizable vinyl compound.

Most commercially available methacrylate and acrylate monomers that can be used in the composition of the invention are supplied with inhibitors to provide those products with good shelf life. Additional inhibitors may be added to the composition to prevent excessive acceleration of the polymerization reaction within the self-cure or dual-cure resin-based composite. This is particularly important in the case of placement of tight-fitting prostheses. The level of inhibitor, if needed, is adjusted to give ample working time during the placement procedure.

Examples of inhibitors useful in the present invention are t-butylhydroxytoluene, hydroquinone, and the methyl ether of hydroquinone. Other useful examples are well known to those familiar with the art. Amounts of added inhibitor in up to 5% by weight of the polymerizable organic material may be found useful in the application of the invention. Preferably less than 2% inhibitors may be required and, indeed, inhibitors present in the commercial monomers may provide all the inhibition needed.

Kits for Adhering Dental Surfaces

A further aspect of the invention provides kits for adhering dental surfaces comprising: (a) a bond enhancer comprising at least one polymerizable monomer or prepolymer, a polymerization accelerator, and an optional polymerization inhibitor, in an organic solvent or mixture of organic solvents; and (b) a light-cure dental adhesive system and/or c) a self-cure or dual-cure dental composite system. In certain preferred embodiments, the kit comprises both a light cure dental adhesive system and a self-cure or dual-cure dental composite. The light-cure dental adhesive system may comprise: 1) a light-cure adhesive; 2) a primer and light-cure adhesive suitable for placement on etched tooth surfaces; 3) a self-etch primer and a light-cure adhesive suitable for placement directly on tooth surfaces; or 4) a light-cure adhesive suitable for placement directly on tooth surfaces without prior priming or etching. Preferably, the self-cure or dual-cure dental composite system is a two-part system.

Kits are preferred that, along with the bond enhancer and the adhesive and/or composite, contain a supply of disposable tips that fit onto the containers, e.g. needles or cannulas, that help to dispense the material onto the biomedical surface. The kits may also comprise one or more application or removal aids, e.g. swabs and/or brushes.

The bond enhancer, light-cure adhesive system and dual-cure or self-cure composite systems are housed in suitable containers. E.g., the light-cure adhesive should be stored in a light-tight container. Similarly, the light-triggered portion of a dual-cure composite system should be stored in a light-tight container. Additionally, the container preferably limits the loss of volatile solvent so as to provide adequate shelf life, e.g., of at least two years. The bond enhancer may be packaged in a single-use or multi-use container.

The kit may also comprise instructions for using the bond enhancer to increase bond strength of the enclosed light-cure dental adhesive to any compatible self-cure or dual-cure composite. Alternatively, the kit may include instructions for using the bond enhancer to increase bond strength of the enclosed self-cure or dual-cure composite to any compatible light-cure adhesive. The kit may include instructions for using the bond enhancer to increase bond strength of the enclosed self-cure or dual-cure composite to the enclosed light-cure adhesive system. Alternatively, the kit may include instructions for using the bond enhancer to increase bond strength of any compatible self-cure or dual-cure composite to any compatible light-cure adhesive. The instructions can be provided with the kit (e.g., instruction material provided in a package together with the kit) or separately (e.g., instruction material provided via a separate booklet, via a video or DVD, via remote access such as the Internet, etc.).

### DETAILED DESCRIPTION OF THE INVENTION

### EXAMPLE 1

### Bond Enhancer Compositions

The components of the bond enhancer compositions (set forth in Table 1) were combined with stirring to effect dissolution into the solvent, forming a clear colorless solution. Controlled heating may effect faster dissolution of solid components. The following abbreviations are used: 3G=triethyleneglycol dimethacrylate, HEMA=2-hydroxyethyl methacrylate, NaBSA=sodium benzenesulfinate, DHEPT=N,N-di(2-hydroxyethyl)-p-toluidine, BHT=t-butylhydroxytoluene, and IPA=isopropanol.

TABLE 1
Bond Enhancer Composition (Weight Percentage)

| | BisGMA | 3G | HEMA | NaBSA | DHEPT | BHT | Ethanol | Acetone | IPA |
|---|---|---|---|---|---|---|---|---|---|
| 1a | 34.3 | | 34.3 | 1.96 | | 0.086 | 29.4 | | |
| 1b | | | | 1.00 | | | | | 99.0 |
| 1c | | | | | 2.00 | | | 98.0 | |
| 1d | 2.2 | | 2.2 | 0.12 | 1.87 | 0.006 | 1.9 | 91.8 | |
| 1e | 1.8 | | 1.8 | 0.10 | 0.40 | 0.004 | 1.5 | 94.5 | |
| 1f | 1.9 | | 1.9 | 0.11 | 0.11 | 0.005 | 1.7 | 94.2 | |
| 1g | 1.8 | | 1.8 | 0.10 | 0.40 | 0.004 | 46.2 | 49.8 | |
| 1h | 2.2 | 2.1 | | 0.13 | 1.00 | 0.006 | 1.9 | 92.7 | |
| 1i | 2.1 | 2.1 | | 0.40 | 1.00 | 0.006 | 39.7 | 54.7 | |
| 1j | 2.1 | 2.1 | | 0.40 | 1.00 | 0.006 | 94.4 | | |
| 1k | 1.1 | 3.2 | | 0.40 | 1.00 | 0.006 | 39.6 | 54.8 | |
| 1l | 1.1 | 3.2 | | 0.40 | 1.00 | 0.006 | 94.3 | | |
| 1m | 2.9 | 1.3 | | 0.40 | 1.00 | 0.006 | 94.4 | | |

### EXAMPLE 2

### Bonding Core Paste to Dentin

Shear bond strength (SBS) testing was accomplished using apparatus and methods described in section 5.2.5 of ISO 11405:2003(E), “Dental materials—Testing of adhesion to tooth structure” and “Test type 1: short-term test after 24 h in water at 37° C.” except that tests were completed after one hour in 100% humidity at 37° C. The apparatus is more fully disclosed in “ISO/TC 106/SC 1/WG 11—Single Plane Shear Test Assembly (Description of Round Robin Test Results), Revised September 1996,” by Larry G. Watanabe, UCSF Dept. of Restorative Dentistry.

Freshly extracted teeth were stored at room temperature in 0.5% Chloramine-T solution for one to three months. Caries free teeth were rinsed, debrided, and stored in tap water under refrigeration for no more than one month prior to use. A diamond-impregnated model trimmer (WhipMix Model 3B) with copious water spray was used to flatten the cusps and roots and remove mesial and distal proximal enamel to expose dentin areas having a diameter of at least 3 mm. The teeth were sectioned at reduced speed using a diamond saw (MTI Corporation Model 250), keeping the blade and tooth wet at all times. The trimmed half teeth were polished on wet 240-grit and then 600-grit sandpaper (3M Wetordry Tri-M-ite) to flat, smooth surfaces, rinsed with tap water, and evaluated at 15× to insure the presence of cut dentin. Finally, the teeth were dragged once across virgin wet 320-grit sandpaper to create a series of furrows similar to those made with diamond burs during clinical practice.

Tooth Specimen Assembly: Mylar tape (TAP Plastics, Dublin, Calif.) with a 3.0±0.1 mm hole was centered over the rinsed and air-dried teeth to form a mask. The mylar was burnished to the specimens to ensure tight adhesion to the surfaces. Each specimen with mylar mask was adhered to the flat surface of the assembly first shear plate by mounting them with the mask hole centered in a 15.6 mm countersunk hole and the furrows created by the 320-grit paper aligned with the force application axis. Each plate was then inverted onto a wet paper towel and die stone (Whip-Mix Flowstone PN 33941), mixed according to the manufacturer's directions to obtain a flowable mixture, was used to fill the cavity in the shear plate around the tooth to a level that covers at least part of a tapered section to lock it in place. After the die stone was set (about 45 minutes), the tooth specimen becomes a rigid component of the shear plate. The second shear plates were securely joined to the first using two brass screws each so that the 5.9 mm countersunk holes are aligned over the holes in the mylar masks.

Bonding system components were applied at 23±1° C. The exposed tooth surface was treated with aqueous 37% phosphoric acid for 15 seconds and rinsed well with water. Prelude Adhesive (Danville Materials) was applied to the slightly moist surface with scrubbing for 10 seconds. It was air-dried with a dental syringe (Henry Schein P N 100-8004) connected to a source of extra dry compressed air (Praxair Cat. No. AI 0.0XD-T) and then cured for 20 seconds using a halogen dental curing light (Demetron Optilux 501) whose output was measured as 700±50 mW/cm². The compositions of the invention were dabbed onto the adhesive and air-dried. Equal portions of Core Paste A and B (Den-Mat) were hand-mixed and applied to the surface from C-R Tubes (Centrix Inc.). The entire assembly was placed in an incubator at 37° C. in 100% relative humidity for one hour prior to bond testing.

Bond Strength Determination—Specimen assemblies were mounted in the universal testing machine (Instron Model 4211) by connecting each shear plate to an alignment apparatus. The alignment apparatus consists of grip adapters attached to the universal tester frame and crosshead grip mounts using pins followed by universal joints and thin alignment plates. The thin alignment plates were attached to the inside surfaces of Plates A and B using centered brass screws so that the force is applied in the same plane as the bond interface. Prior to debonding, the two brass screws that hold the plates together are removed so that the bond interface is the only link between the plates. A small pretension force of 8±3 newtons was applied 15±5 seconds prior to commencement of loading. Force was applied at a constant speed of 1 mm/minute until bond failure was observed and recorded. The Instron Series IX software calculates the shear bond strength, SBS, or peak strength, in MPa (megapascals) as:
SBS=F/A
where:
F=Load at break in Newtons
A=Cross sectional area of the bonded surface in mm²

Table 2 provides the resulting shear bond strength for each bond enhancer composition of Example 1 to etched dentin when using a typical light-cure dental adhesive, Prelude, and a self-cure composite, Core Paste. Without using the composition of the invention, Core Paste has no adhesion to light-cured Prelude Adhesive on etched dentin. Each data point represents the mean value of five specimen bond strengths.

TABLE 2
Bond Strength

| Bond Enhancer | SBS (MPa) |
|---|---|
| 1a | 16.00 |
| 1b | 13.60 |
| 1c | 2.80 |
| 1d | 22.30 |
| 1e | 23.70 |
| 1f | 18.80 |
| 1g | 29.80 |
| 1h | 30.37 |
| 1i | 31.58 |
| 1j | 34.23 |
| 1k | 32.99 |
| 1l | 23.87 |
| 1m | 25.36 |

### EXAMPLE 3

### Bond Enhancer Compositions

The components of the bond enhancer (set forth in Table 3) were combined with stirring to effect dissolution into the solvent, forming a clear colorless solution.

TABLE 3
Bond Enhancer Composition (Weight Percentage)

| | BisGMA | 3G | NaBSA | DHEPT | BHT | Ethanol |
|---|---|---|---|---|---|---|
| 2a | 2.1 | 2.1 | 0.4 | 1.0 | 0.006 | 94.4 |
| 2b | 2.1 | 2.1 | 0.4 | 1.0 | 0.024 | 94.4 |
| 2c | 2.1 | 2.1 | 0.4 | 1.0 | 0.037 | 94.4 |
| 2d | 2.1 | 2.1 | 0.4 | 1.0 | 0.050 | 94.4 |
| 2e | 2.1 | 2.1 | 0.4 | 1.0 | 0.060 | 94.3 |
| 2f | 2.1 | 2.1 | 0.1 | 0.4 | | 95.3 |
| 2g | 2.1 | 2.1 | 0.1 | 0.6 | | 95.1 |
| 2h | 2.1 | 2.1 | 0.2 | 0.6 | | 95.0 |
| 2i | 2.1 | 2.1 | 0.5 | 1.2 | | 94.1 |
| 2j | 2.1 | 2.1 | 0.5 | 1.4 | | 93.9 |
| 2k | 2.1 | 2.1 | 0.1 | 0.4 | 0.01 | 95.3 |
| 2l | 2.1 | 2.1 | 0.4 | 1.0 | 0.01 | 94.4 |
| 2m | 2.1 | 2.1 | 0.1 | 0.4 | 0.04 | 95.3 |
| 2n | 2.1 | 2.1 | 0.4 | 1.0 | 0.04 | 94.4 |

### EXAMPLE 4

### Working Time

Prelude Adhesive is applied to the center of two clean 1″×3″ glass slides, air-dried, and cured with an Optilux 501 curing unit for 20 seconds. The composition of the invention is dabbed onto the adhesive surface and air-dried. The working time is determined by placing approximately 30 mgs of mixed StarFill 2B (Danville Materials) paste onto one of the treated slides, starting a timer at the moment the mixed paste is discharged from its automix tip. The other treated glass slide is gently placed crosswise to the original slide so that StarFill 2B is sandwiched between the treated adhesive layers. The assembly is transferred to a 37° C. incubator oven within 15 seconds of the start time. At a prescribed exposure time, the upper glass slide is pressed in order to flatten the mixture. If the working time has been exceeded, polymerization will be evidenced by clefts and voids at the periphery of the spreading paste or by crystalline masses within it. If polymerization has not occurred, the paste will flow to fill the intersection between the glass slides. The test is repeated until the working time is bracketed within 5 seconds, that is, at the shorter exposure time no sign of polymerization is evidenced, but an exposure of 5 seconds longer duration shows signs of polymerization of the paste.

TABLE 4
Working Time with Bond Enhancer

| Bond Enhancer | Working Time, s |
|---|---|
| 2a | 65.0 |
| 2b | 77.5 |
| 2c | 82.5 |
| 2d | 87.5 |
| 2e | 87.5 |
| 2f | 82.0 |
| 2g | 72.5 |
| 2h | 70.0 |
| 2i | 60.0 |
| 2j | 55.0 |
| 2k | 77.0 |
| 2l | 65.0 |
| 2m | 91.0 |
| 2n | 82.5 |

In 2a through 2e, as the weight percent of BHT, an inhibitor, increased from 0.006% to 0.060% while keeping all other component levels the same, the working time increased from 65 seconds to 87.5 seconds. Comparing 2f to 2g and 2i to 2j shows that increasing the amount of DHEPT shortens the working time. Comparing 2k to 21 and 2m to 2n shows that increasing the total polymerization accelerators decreases the working time.

### EXAMPLE 5

### Adhesion to Various Dental Surfaces

Shear bond strengths were determined by the Watanabe Method Shear Bond Strength Test method as described in Example 2 except that various other dental surfaces, each with their own specific preparation protocol, were used. Instances where the bond enhancer composition 1j, above, was used were compared to instances without its use. Commercial adhesives were applied according to their manufacturer's instructions except in specified instances where the adhesive was not light-cured. Specimens were stored for one hour at 37° C. and 100% R.H. prior to force application.

Untreated Dentin: Clearfil S³ Bond (Kuraray Medical) was applied directly to dentin according to the manufacturer's instructions and light-cured. In the first test, StarFill 2B was applied to the adhesive surface directly through a static automix tip and not subsequently light-cured resulting in a shear bond strength of 3.51 MPa after one hour. StarFill 2B is a flowable dual-cure resin-based composite dental restorative. In the next test, the bond enhancer was applied as described in Example 2 and StarFill 2B was applied to the treated adhesive surface directly through a static automix tip and not subsequently light-cured resulting in a shear bond strength of 19.62 MPa after one hour. In the last test, Accolade (Danville Materials), a light-cure flowable resin-based dental restorative, was applied to the surface through a cannula and light-cured to obtain a shear bond strength of 19.17 MPa at one hour age. Clearfil S³ Bond, a single-component light-cure dental bonding system, is not indicated for use with self-cure composites or dual-cure composites. Clearfil S3 Bond contains 10-methacryloyloxydecyl dihydrogen phosphate, BisGMA, HEMA, hydrophobic dimethacrylate, dl-camphorquinone, ethanol, water, and silanated colloidal silica. The resulting bond strengths, provided in Table 5, demonstrate that bond enhancer improves the bond strength of StarFill 2B to Clearfil S³ Bond on dentin. The strength obtained is similar to when Clearfil S3 Bond is applied to dentin and, in accordance with its indicated use, is covered with Accolade, a light-cure composite.

TABLE 5
Bond Strength to Unreacted Dentin

| | Adhesive | | Composite | |
|---|---|---|---|---|
| Adhesive | Cure Type | Bond Enhancer | Brand | Bond Strength |
| Clearfil S³ | Light-Cure | None | StarFill 2B | 3.51 MPa |
| Bond | | | (self-cure) | |
| Clearfil S³ | Light-Cure | 1j | StarFill 2B | 19.62 MPa |
| Bond | | | (self-cure) | |
| Clearfil S³ | Light-Cure | None | Accolade | 19.17 MPa |
| Bond | | | (light-cure) | |

Primed Dentin: Primers were applied to dentin surface with a fuzz-tipped micro-applicator brush according to manufacturer's instructions. The primers were not rinsed. An air syringe was used to gently evaporate the solvent. Adhesives were applied according to manufacturer's instructions. The bond enhancer and Core Paste, a self-cure core build-up composite, were applied as described in Example 2. StarFill 2B was applied to the surface directly through a static automix tip and not subsequently light-cured. The resulting bond strengths, provided in Table 6, demonstrate that the bond strength is independent of whether or not the Prelude Adhesive layer is light cured when bond enhancer is used. Without light curing, Prelude Adhesive with the composition of the present invention bonds StarFill 2B (self-cure only) to primed dentin much better than Optibond Solo plus Adhesive (Kerr SDS) mixed with its Activator (self-cure only).

TABLE 6
Bond Strength to Primed Dentin

| | | | Self-Cure | |
|---|---|---|---|---|
| Primer and | Adhesive | Bond | Composite | |
| Adhesive | Cure Type | Enhancer | Brand | Bond Strength |
| Prelude | Light-Cure | 1j | Core Paste | 29.05 MPa |
| Prelude | No Cure | 1j | Core Paste | 28.85 MPa |
| Prelude | No Cure | 1j | StarFill 2B | 21.85 MPa |
| Optibond Solo | Self-Cure | none | StarFill 2B | 5.08 MPa |
| plus SE | | | | |

Etched Dentin: Sure Etch Liquid (Danville Materials) was applied to dentin surface with a micro-applicator. After 10 seconds, the etchant was rinsed from the dentin. The dentin was then dabbed with a micro-applicator to leave some residual moisture. The adhesive, bond enhancer and composites were applied as described previously for primed dentin. The resulting bond strengths, provided in Table 7, demonstrate that Prelude Adhesive bonds various restoratives to etched dentin equally well without light curing at any step when used with bond enhancer of the present invention.

TABLE 7
Bond Strength to Etched Dentin

| | Adhesive | | Self-Cure | |
|---|---|---|---|---|
| | Cure | Bond | Composite | Bond |
| Adhesive | Type | Enhancer | Brand | Strength |
| Prelude | Light-Cure | 1j | Core Paste | 30.97 MPa |
| Prelude | No Cure | 1j | Ti-Core auto E | 22.77 MPa |
| Prelude | No Cure | 1j | Ti-Core Natural | 20.47 MPa |
| Prelude | No Cure | 1j | Flexi-Flow | 26.62 MPa |
| | | | Natural | |
| Prelude | No Cure | 1j | Flexi-Flow Cem | 23.19 MPa |
| Optibond | Dual-Cure | none | Core Paste | 14.90 MPa |
| Solo plus | | | | |

Ti-Core and Flexi-Flow are products of Essential Dental Systems

Empress Porcelain: The porcelain surface was air abraded using 50p alumina (Danville Materials) at 70 psi. 9.6% HF Gel (Pulpdent) was applied and allowed to sit for 60 seconds and then completely rinsed from the porcelain. The porcelain was thoroughly dried prior to application of Bond Star S (Danville Materials), which was permitted to sit for 10 seconds prior to drying. The adhesive, bond enhancer and composites were applied as described previously for primed dentin. The resulting bond strengths, provided in Table 8, demonstrate that bond strength to porcelain using StarFill 2B (self-cure only) as restorative is independent of whether or not Prelude Adhesive layer is light cured when a composition of the present invention is used.

TABLE 8
Bond Strength to Porcelain

| | | | Self-Cure | |
|---|---|---|---|---|
| | Adhesive Cure | | Composite | Bond |
| Adhesive | Type | Bond Enhancer | Brand | Strength |
| Prelude | Light-Cure | 1j | StarFill 2B | 24.88 MPa |
| Prelude | No Cure | 1j | StarFill 2B | 25.02 MPa |

Rexillium IV: The Rexillium surface was abraded using 50 alumina at 70 psi and subsequently sonicated in water for 10 minutes. After drying, adhesive, bond enhancer and composites were applied as described previously for primed dentin. The resulting bond strengths, provided in Table 9 demonstrate that without light curing, the bond strength of StarFill 2B to Rexillium IV using Prelude Adhesive with a composition of the invention is 21.0% lower than when the adhesive is cured. Even still, bond enhancer used with uncured Prelude Adhesive is better than using no adhesive and is similar to using Panavia (Kuraray) as restorative.

TABLE 9
Bond Strength to Rexillium IV

| | Adhesive | | Self-Cure | |
|---|---|---|---|---|
| | Cure | Bond | Composite | |
| Adhesive | Type | Enhancer | Brand | Bond Strength |
| Prelude | Light-Cure | 1j | StarFill 2B | 16.84 MPa |
| Prelude | No Cure | 1j | StarFill 2B | 13.27 MPa |
| None | na | None | StarFill 2B | 10.09 MPa |
| None | na | None | Panavia | 12.76 MPa |
| Prelude | Light-Cure | 1j | Core Paste | 17.50 MPa |
| Optibond Solo | Light-Cure | 1j | Core Paste | 11.43 MPa |
| plus | | | | |

na = not applicable

Penceram 62% Gold: The Penceram surface was air abraded using 50 μ alumina at 70 psi and subsequently sonicated in water for 10 minutes. After drying, Tin-plate (Danville Materials) was applied and allowed to sit for 3 seconds prior to rinsing. After drying, adhesive, bond enhancer and composites were applied as described for primed dentin. The resulting bond strengths, provided in Table 10, demonstrate that without light curing, the bond strength of StarFill 2B to Penceram 62 using Prelude Adhesive with a composition of the invention is 20.4% lower than when the adhesive is cured. Even still, Prelude Adhesive without curing the adhesive is better than using no adhesive or when using Panavia as restorative.

TABLE 10
Bond Strength to Metal

| | Adhesive | | Self-Cure | |
|---|---|---|---|---|
| | Cure | | Composite | |
| Adhesive | Type | Bond Enhancer | Brand | Bond Strength |
| Prelude | Light-Cure | 1j | StarFill 2B | 22.35 MPa |
| Prelude | No Cure | 1j | StarFill 2B | 17.78 MPa |
| None | na | None | StarFill 2B | 14.34 MPa |
| None | na | None | Panavia | 14.67 MPa |

na = not applicable

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A bond enhancer for placement between a light-cure adhesive and a self-cure or dual-cure composite, said enhancer comprising at least one polymerizable monomer or prepolymer and a polymerization accelerator in an organic solvent or mixture of organic solvents.

2. The bond enhancer of claim 1, further comprising a polymerization inhibitor.

3. The bond enhancer of claim 1, wherein the organic solvent is selected from the group consisting of acetone, acetonitrile, benzyl alcohol, 1-butanol, 2-butanol, isobutanol, tert-butanol, chloroform, ethanol, ethyl acetate, ethyl methyl ketone, methyl acetate, 1-propanol, and 2-propanol, and mixtures thereof.

4. The bond enhancer of claim 3, wherein the organic solvent is ethanol.

5. The bond enhancer of claim 1, wherein the organic solvent is present in the bond enhancer at a level of from about 10% to about 99.5% by weight.

6. The bond enhancer of claim 5, wherein the organic solvent is present in the bond enhancer at a level of from about 90% to about 98% by weight.

7. The bond enhancer of claim 1, wherein the polymerizable monomer is a methacrylate compound.

8. The bond enhancer of claim 7, where the methacrylate compound is Bowen monomer bis-GMA, ethoxylated Bis-GMA, triethyleneglycol dimethacrylate, or mixtures thereof.

9. A method of bonding a light-cure adhesive to a self-cure or dual-cure composite comprising:
(a) applying a bond enhancer to a layer of light-cure adhesive, wherein the bond enhancer comprises at least one polymerizable monomer or prepolymer and a polymerization accelerator in an organic solvent or mixture of organic solvents; and
(b) applying the self-cure or dual-cure composite to the bond enhancer prior to the self-cure or dual-cure composite being fully cured.

10. The method of claim 9, wherein the bond enhancer is applied to the layer of light-cure adhesive before the light-cure adhesive is cured.

11. The method of claim 9, wherein the bond enhancer is applied to the layer of light-cure adhesive after the light-cure adhesive has been exposed to a light source.

12. A method of enhancing a bond to a biomedical surface comprising:
(a) applying a light-cure adhesive to a biomedical surface;
(b) applying a bond enhancer to the light-cure adhesive, wherein the bond enhancer comprises at least one polymerizable monomer or prepolymer and a polymerization accelerator in an organic solvent or mixture of organic solvents; and
(c) applying a self-cure or dual-cure composite to the bond enhancer prior to the self-cure or dual-cure composite being fully cured.

13. The method of claim 12, further comprising applying an etchant to the biomedical surface prior to applying the light-cure adhesive.

14. The method of claim 13, further comprising applying a primer the biomedical surface prior to applying the light-cure adhesive.

15. The method of claim 14, wherein at least two of the etchant, primer and light-cure adhesive are combined into a single composition.

16. The method of claim 12, further comprising light curing the light-cure adhesive prior to applying the bond enhancer.

17. A method of enhancing a bond between two biomedical surfaces comprising:
(a) applying a light-cure adhesive to a first biomedical surface;
(b) applying a bond enhancer to the light-cure adhesive, wherein the bond enhancer comprises at least one polymerizable monomer or prepolymer and a polymerization accelerator in an organic solvent or mixture of organic solvents;
(c) applying a self-cure or dual-cure composite to a second biomedical surface; and
(d) fitting the second biomedical surface to the first biomedical surface, prior to the self-cure or dual-sure composite being fully cured.

18. The method of claim 17, further comprising applying a second light-cure adhesive to the second biomedical surface and applying a second bond enhancer to the second light-cure adhesive, prior to applying the self-cure or dual-cure composite to the second biomedical surface.

19. The method of claim 17, further comprising applying an etchant to the first or second biomedical surface prior to applying the light-cure adhesive.

20. The method of claim 19, further comprising applying a primer the biomedical surface prior to applying the light-cure adhesive.

21. The method of claim 20, wherein at least two of the etchant, primer and light-cure adhesive are combined into a single composition.

22. The method of claim 17, further comprising light curing the light-cure adhesive prior to applying the bond enhancer.

23. A kit for adhering dental surfaces comprising:
(a) a bond enhancer comprising at least one polymerizable monomer or prepolymer and a polymerization accelerator in an organic solvent or mixture of organic solvents; and
(b) a light-cure dental adhesive system, or a self-cure or dual-cure dental composite.

24. The kit of claim 23, wherein the bond enhancer further comprises a polymerization inhibitor.

25. The kit of claim 23, comprising a light-cure dental adhesive.

26. The kit of claim 23, comprising a self-cure or dual-cure dental composite.

27. The kit of claim 23, comprising both a light cure dental adhesive system and a self-cure or dual-cure dental composite.

28. The kit of claim 23, wherein the light-cure dental adhesive system comprises
1) a light-cure adhesive;
2) a primer and light-cure adhesive suitable for placement on etched tooth surfaces;
3) a self-etch primer and a light-cure adhesive suitable for placement directly on tooth surfaces; or
4) a light-cure adhesive suitable for placement directly on tooth surfaces without prior priming or etching.
